(19) United States
(12) Patent Application Publication
(10) Pub. No.: US 2020/0230185 A1
(43) Pub. Date: Jul. 23, 2020

(54) COMPOSITION CONTAINING CANNABINOIDS WITH IMPROVED BIOAVAILABILITY

(71) Applicant: CRESO PHARMA SWITZERLAND GMBH, Cham (CH)

(72) Inventors: Miriam Halperin Wernli, Baar (CH); Jorge Wernli, Baar (CH); Gian Batista Trepp, Fribourg (CH); Silvia Huber, Lengnau (CH); Franco Madea, Lengnau (CH)

(21) Appl. No.: 16/637,183

(22) PCT Filed: Aug. 7, 2018
(86) PCT No.: PCT/IB2018/000862

§ 371 Date: Feb. 6, 2020

Related U.S. Application Data
(60) Provisional application No. 62/542,459, filed on Aug. 8, 2017.

(51) Int. Cl. A61K 36/185; A23L 33/105

(52) U.S. Cl. CPC A61K 36/185; A61K 9/0056; A23L 33/105

(57) ABSTRACT

A food or nutritional or dietary composition or food or nutritional or dietary supplement suited for buccal delivery of cannabinoids comprises a source of cannabinoids in combination with a vasodilator agent selected from essentials oils or components of same, flavors or components of same and spices or components of same. Preferred source of cannabinoids is standardized oil or extract of hemp (Cannabis sativa L.).

### INTRODUCTION

Administering a medicine, a drug or any other active substance is often done orally. Tobacco and nicotine, cannabis and cannabinoids such as THC, CBD, CBG etc., are smoked and the basic plant material active ingredients are inhaled and the smoke contains the desired active substances. The switch to the use of an electronic cigarette, on the other hand, requires the use of concentrated or purified extracts of vegetal material or solutions of said active substances.

In the field of medical application of active substances, oral administration can be used but by means of more elaborated release formulations suitable e.g. for a sublingual deposit or via contact with buccal mucosa like e.g. adhesive films, granules, tablets, gels, oral sprays, etc. US 2005/257,463; US 2016/058.866 and many others as well are disclosing such formulations.

However, oral administration of medicinal or other active substances encounters frequently the barrier of the oral mucosa of the subject in need thereof, which by way of consequence slows down the absorption of the said substance, in other words the passing of the active substance from the external medium e.g. the saliva or the gastrointestinal fluid to the bloodstream.

This phenomenon may reduce substantially the bioavailability of said active substance, in other words the fraction of active substance which is actually absorbed by the organs and reaches the bloodstream.

This applies in particular to active substances such as tobacco alkaloids like nicotine or e.g. hemp or cannabis cannabinoids (THC, CBD, CBG, etc.), whether these materials are reserved for purely recreational use or for medical use. In such cases and in order to obtain the desired effect, it is important that the latter be rapidly transported by the bloodstream, and in optimum concentration, to the relevant endocannabinoid receptors dispersed in the body and especially to the brain.

Various solutions have already been proposed in order to overcome such difficulties, with particular attention being paid to obtaining the best-suited and best-performing release formulation, such as an emulsion described in US Pat. No. 2016/058,866.

Another solution consists in either modifying the molecular structure of the selected active substance, for example making it more lipophilic or hydrophilic, or subjecting it to nano-encapsulation or vectorizing. This constitutes, however, a modification of the molecular structure that can lead sometimes to negative or uncontrolled metabolic side effects.

Still another solution consists in incorporating into the above-mentioned release formulation permeation or penetration agents, i.e. ingredients that shall enhance absorption through the mucosal coating or epithelium (permeability enhancement). These agents, usually, interact with the cellular membrane of the mucosa without interfering with the molecular structure of the active substance at stake. US2014/166,027 discloses such permeation agents, which are essentially synthetic chemicals such as polyethylene glycols (PEG), diethylene-glycol ethers like TRANSCUTOL® or EDTA, bile salts, synthetic surfactants, etc. The majority of the permeation agents disclosed therein, however, is designed for transdermal administration and many of same are neither GRAS nor even edible.

US2014/166,027 also envisages the use of “thermal agents”, the expected effect of which is to increase blood flow which can in turn increase absorption. US 2014/166,027 disclosure, however, does not cite any particular substance and merely refers to the use of chemicals causing an exothermic reaction in situ. Such a heat release, unfortunately, is perceived negatively by the subject at stake, namely as “non-natural” if not frightening and this has led the authors to avoid using these means of oral administration.

Experience further shows that the solutions provided in US 2014/166,027 are hardly compatible with the administration of a drug or recreational active substance such as THC, CBD, CBG, etc., especially due to the generation of unpleasant taste or flavors, or off taste and off flavors.

The object of the invention is precisely to provide a solution which makes it possible to overcome or wipe out advantageously the drawbacks and negative elements listed above.

### THE INVENTION

The composition according to the invention consists in a food or nutritional or dietary composition or a food or nutritional or dietary supplement suited for buccal delivery of cannabinoids which comprises a source of cannabinoids in combination with a vasodilator agent selected from essential oils or components of same, flavors or components of same and spices or components of same.

A suitable source of cannabinoids according to the invention is standardized oil or extract of hemp (Cannabis sativa L.), preferably standardized oil or extract of hemp (Cannabis sativa L.) comprising minor amounts of psychotropic substances.

The composition most frequently met within the frame of the invention is typically a standardized dosage unit customarily used in the pharmaceutical and/or confectionery industries.

### Definitions

The term “essential oil” defines vegetal oils or extracts like those used in the food or flavor industry and that are employed for preparing “hot” sauces, “hot” condiments or “hot meals. These include but are not restricted to capsicum oil, clove oil, mustard oil, etc. The flavor or pharmaceutical industry provides commercially available essential oils or extracts of same that can be conveniently used within the frame of the invention.

The term “flavor component” defines ingredients and mixtures of ingredients either of natural or synthetic origin conventionally used in the industry, and in particular for preparing “hot” sauces, condiments or meals. These include but are not restricted to sinigrin isolated from mustard seeds, capsaicin isolated from chili pepper extract or piperin isolated from black pepper extract. The flavor industry provides commercially available powders (e.g. spay dried dextrin-based powders) or liquid extracts that can be conveniently used within the frame of the invention.

The term “spice” defines spices usually employed during everyday life either for cooking or confectionery like e.g. black pepper, chili pepper, ginger, ginseng, mustard, horse radish, curcuma, cardamom, cumin and the like. The latter are most frequently applied in their standardized powdered form or, whenever possible as solution in edible oil. They are commercially available and can be conveniently used within the frame of the invention.

The term “chewing gum” defines edible chewable compositions that do not disintegrate during chewing wherein active substances or flavor components are dispersed within the gum-based matrix or are put in a liquid core (reservoir) surrounded by the gum base.

The term “film” defines a release system such as a thin sheet of edible material that dissolves or disintegrates rapidly if not immediately when in contact with saliva and which releases simultaneously the cannabinoids, or any additional active substance contained therein, into the buccal cavity.

This term covers also sublingual or muco-adhesive films like those met for conventional administration of medicaments like e.g. those made of water-soluble polymers incorporating the selected active substance and coated with bio-adhesive made of e.g. HPC, hydroxyl-propyl-cellulose.

The term “capsules” cover hard and soft edible capsules like those met in the art for the administration of conventional recreational or therapeutic agents or food or nutritional ingredients or micronutrients.

The term “granules” defines here edible particles in their granulated form, e.g. such as spherical or similar particles consisting essentially of a biodegradable matrix containing the selected active substances, the cannabinoids extracted from hemp or cannabis dispersed inside.

The term “paste” defines more or less compact or viscous edible pasty material that can be chewed over a certain period, i.e. necessary for the buccal delivery of the active substance and eventually swallowed. Examples of same are fruits pastes, gums, soft gums, tooth paste and the like.

The term “tablet” defines either soft or hard tablets such as those met in the pharmaceutical industry and in particular compressed tablets, molten sugar-based tablets, extruded tablets and the like.

The term “candies” defines any food composition usually provided to subjects like teenagers or adults for either recreational use or for enabling them an easy and pleasant absorption of active ingredients like medicaments, mouth refreshing agents, mouth deodorants and the like. The candies most frequently met within the frame of the invention are soft gums or granules or tablets.

The term “with minor amounts of” define here cannabis or hemp (Cannabis sativa L.) oils or extracts wherein the content of psychotropic substances, namely THC is significantly below the applicable regulatory provisions according to EFSA and EU regulatory bodies recommendation to be equal to or lower than 1 microgram per kg, usually below 0.05% (weight) of the relevant extract.

Some of such oils or extracts are commercially available whereas others can be obtained when subjecting the adequate raw plant material i.e. buds, leaves, flowers, etc. or their oils or their extracts to specific extraction and purification techniques.

The terms “standardized oil” and “standardized extract” apply to compositions of matter that are either already commercially available or manufactured on request by dedicated companies that are mastering current extraction, e.g. solvent or CO2 extraction, and purification techniques and that can deliver oils or extracts with a precisely defined and characterized content in active substances, cannabinoids in the instant case.

Standardized cannabis or hemp (Cannabis sativa L.) oil or extracts defined here above are quite convenient for preparing compositions according to the invention designed for either therapeutic or nutritional or pure recreational use.

Hemp (Cannabis sativa L.) extracts that can be conveniently used within the frame of this invention present generally a high content in non-psychotropic cannabinoids and in particular CBD, namely up to ca. 25% or even, exceptionally as high as 60 to 80%, whereas the content of psychotropic substances, in particular THC, ranges most generally from 0.01 up to 0.2%. Proportions of THC, exceptionally, can even be moved up to any legally or regulatory authorized level, depending on the application which is selected and the local context.

An example of such a preferred standardized hemp extract comprises 23.6% CBD, 0.25% of a mix of CBDV, CBDA & CBG and less than 0.05% THC.

### EMBODIMENTS

Manufacturing any of the above embodiments of the food or dietary compositions of the invention does not require any specific or complex technology and can conveniently apply the usual confectionary procedures.

The proportions of active ingredients, i.e. the cannabinoids present in the hemp extracts, essentially CBD, and the vasodilator agent in the final food or dietary composition or supplement shall greatly vary depending on the concentration of CBD in the said hemp extracts to select as well as that of e.g. capsaicin in the chili pepper or capsicum extract selected as the source of vasodilator agent.

Sources of such vasodilator agents vary extensively and can comprise essential oils selected from capsicum oil, clove oil or mustard oil; flavors components selected from to sinigrin isolated from mustard seeds or capsaicin isolated from chili pepper extract or piperine isolated from black pepper; spices selected from black pepper, chili pepper, ginger, ginseng, mustard, horse radish, curcuma, cardamom or cumin. This enumeration, however, is not limitative.

The man skilled in the art shall have full choice of combinations or ratios of “vasodilatory agent versus cannabinoids” when making use of commercially available products in order to achieve the desired effect, for instance a fast or moderate speed of cannabinoid buccal absorption or a strong or moderate metabolic effect due to the CBD concentration in blood stream. Convenient ratios within the combination of “vasodilator agent to CBD” shall range from 1:5′000 to 1:500 (weight).

In one of the particular embodiments of the invention, when making use of a hemp extract containing ca. 23-25% CBD (see above) and of a capsicum extract containing ca. 2% capsaicin good performances have been achieved using weight ratios of “vasodilatatory agent to CBD” of around 1:3′000-1:2′000.

### EXAMPLE(S)

The following ones are mere illustration of the invention and they are not deemed to represent any restriction of same.

### Example 1

Gum Based Lozenges have been Prepared by Means of the Following Ingredients (Amounts Provided in Grams):

| | |
|---|---|
| Sorbitol syrup | 34.00 |
| Maltol syrup | 7.40 |
| Gum Arabic | 44.00 |
| Hemp extract* | 2.10 |
| Citric acid | 1.50 |
| Vitamins and minerals mix** | 1.60 |
| Flavoring agent (lime flavor) | 0.25 |
| Sweeteners (sucralose & acesulfame K) | 0.11 |
| Capsicum extract*** | 0.01 |
| Glazing agent (bees wax) | 1.00 |
| Water | q. add to 100 |

*standardized hemp extract comprises 23.6% CBD, 025% of a mix of CBDV, CBDA & CBG and less than 0.05% THC
**Vit. C; Niacine; Vit B; Vitamin B6; Vitamin B12; Zinc
***standardized commercial capsicum liquid extract containing 2.2% capsaicin.

### Example 2

Compressed Sugar-Based Tablets have been Prepared Using the Following Ingredients (Amounts Provided in Grams):

| | |
|---|---|
| Sucrose monolaurate | 16.00 |
| Lactose | 75.20 |
| Ascorbic acid | 0.80 |
| Hemp extract* | 8.00 |
| Powdered lime flavor | 0.25 |
| Capsicum extract*** | 0.01 |

to afford 100 g of dry granules which were then directly compressed into tablets suited for per oral administration as follow ingredients (amounts provided in grams):

| | |
|---|---|
| Granules resulting from above | 75.00 |
| Lactose | 23.80 |
| Magnesium stearate | 1.00 |
| Silicone dioxide (anhydrous) | 0.20 |

*standardized hemp extract comprises 23.6% CBD, 0.25% of a mix of CBDV, CBDA & CBG and less than 0.05% THC
***standardized commercial capsicum liquid extract (contains 2.2% capsaicin).

### Example 3

A fast dissolving sublingual film formulation has been prepared following conventional techniques, making use of polyvinyl alcohol (PVA) as base polymer material, polyvinyl pyrrolidone (PVP) as plasticizer. Mannitol was added as a sweetener in certain examples together with lime flavor (see Example 1).

The proportions and the ratios of Hemp extract and capsicum extracts used for manufacturing such film formulations were identical to those disclosed in Example 1.

1. A food or nutritional or dietary composition or a food or nutritional or dietary supplement suited for buccal delivery of cannabinoids comprising a source of cannabinoids in combination with at least one vasodilator agent selected from the group consisting of essential oils or components of same, flavors or components of same and spices or components of same.

2. The food or nutritional or dietary composition or food or nutritional or dietary supplement according to claim 1, wherein the source of cannabinoids comprises oil or an extract of hemp (Cannabis sativa L.).

3. The food or nutritional or dietary composition or food or nutritional or dietary supplement according to claim 1, wherein the source of cannabinoids is oil or an extract of hemp (Cannabis sativa L.) with minor amounts of psychotropic substances.

4. The food or nutritional or dietary composition or food or nutritional or dietary supplement according to claim 1, wherein the source of cannabinoids is a solvent extract of hemp (Cannabis sativa L.) comprising at least ca. 25% (weight) of CBD.

5. The food or nutritional or dietary composition or food or nutritional or dietary supplement according to claim 1, wherein the essential oils are selected from the group consisting of capsicum oil, clove oil, mustard oil, fenugreek oil and mixtures thereof.

6. The food or nutritional or dietary composition or food or nutritional or dietary supplement according to claim 1, wherein the flavors or components of same are selected from the group consisting of sinigrin isolated from mustard seeds, capsaicin isolated from chili pepper extract, piperin from black pepper extract and mixtures thereof.

7. The food or nutritional or dietary composition or food or nutritional or dietary supplement according to claim 1, wherein the spices are selected from the group consisting of black pepper, chili pepper, ginger, ginseng, mustard, horse radish, curcuma, cardamom, cumin and mixtures thereof.

8. The food or nutritional or dietary composition or food or nutritional or dietary supplement according to claim 1, wherein the source of cannabinoids comprises hemp extract, and the weight ratio of the at least one vasodilator agent to the hemp extract is between 1:5′000 and 1:500.

9. The food or nutritional or dietary composition or food or nutritional or dietary supplement according to claim 1, in a form selected from the group consisting of chewing gums, films, granules, gums, lozenges, candies, pastes and tablets.
